(19) United States
(12) Patent Application Publication
(10) Pub. No.: US 2011/0151476 A1
(43) Pub. Date: Jun. 23, 2011

(54) METHOD OF DETECTION AND/OR TITRATION IN VITRO OF AN UNCONVENTIONAL TRANSMISSIBLE AGENT

(75) Inventors: Benoit Flan, Limours (FR); bruno You, L'hay Les Roses (FR)

(73) Assignees: LFB-Biotechnologies, Les Ulis (FR); Institut National de la Recherche Agronomique, Paris Cedex 07 (FR)

(21) Appl. No.: 12/933,492

(22) PCT Filed: Mar. 25, 2009
(86) PCT No.: PCT/FR2009/050520

§ 371 Date: Sep. 20, 2010

(30) Foreign Application Priority Data
Mar. 27, 2008 (FR) 0851998

(51) Int. Cl. G01N 33/53; C12Q 1/02

(52) U.S. Cl. 435/7.1; 435/29

(57) ABSTRACT

The invention relates to an in vitro method for the in vitro detection and/or titration of a non-conventional transmissible agent (NCTA) or of a protein of pathological conformation, which is a marker for infectiousness related to the NCTA, in a sample, comprising: replication or propagation, in cells in culture, of the NCTA present in the sample, and then repeated incubation with a substrate which allows amplification of the NCTA or of the protein of pathological conformation, nonpathological conformer of the NCTA, before determination of the presence and/or of the amount of the NCTA or of the protein of pathological conformation in the sample.

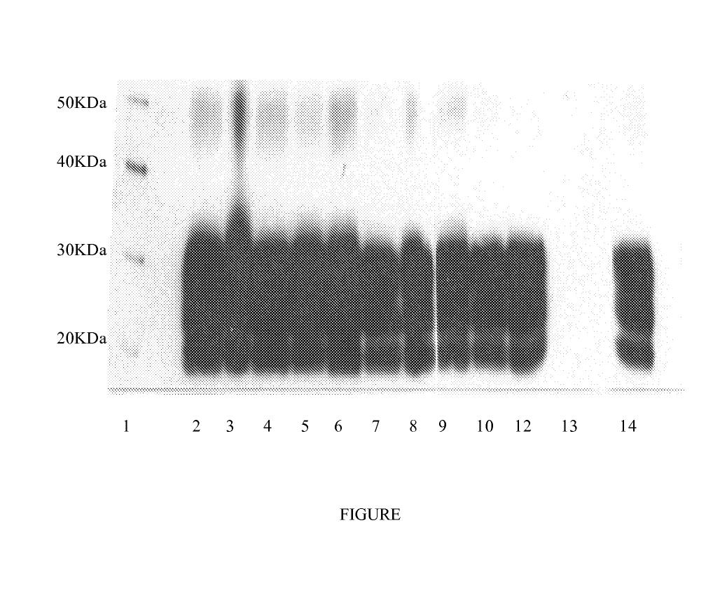

The present invention relates to a method for the in vitro determination of infectiousness related to non-conventional transmissible agents (NCTAs), and to the application thereof, in particular in a method for the in vitro evaluation and/or monitoring of the effectiveness of a method for obtaining or treating a biological product or in a method for the in vitro evaluation and/or monitoring of a decontamination procedure or in a method for the in vitro evaluation of the ability of a compound to modulate NCTA-related infectiousness.

### PRIOR ART

Transmissible spongiform encephalopathies (TSEs) group together a collection of genetic or acquired diseases characterized by degeneration of the central nervous system (CNS). The most common form in humans is Creutzfeldt-Jakob disease (CJD), but TSEs also exist in many mammals (in particular scrapie in sheep and bovine spongiform encephalopathy). The etiological agent of these diseases is classified in the category of “non-conventional transmissible agents” (NCTAs). The signature of the disease is the presence of an extracellular protein, called prion protein (PrP), which is converted during the disease into an insoluble form that is resistant to proteases such as proteinase K, and which accumulates in the central nervous system. This pathological abnormal form of PrP, called PrPsc, copurifies with infectiousness and its accumulation precedes the appearance of histological lesions. It results from a modification of the conformation of the PrP prion protein. No modification of the expression of the gene encoding PrP has been demonstrated, nor has any impairment of its translation (Prusiner, Biochemistry 1992; 31; 12277-88).

The data currently available do not make it possible to demonstrate that the transmissible agent responsible for TSEs is found in an infecting form in blood derivatives (Brown et al., 2001, Semin Hematol.; 38 (4 Suppl 9): 2-6).

However, it cannot be concluded that it is absent, this uncertainty resulting, firstly, from the probable very low concentration in the blood and, secondly, from the very long clinically silent incubation period characteristic of these diseases, which precedes the appearance of clinical signs.

In addition, the exceptional resistance of NCTAs prevents recourse to methods of inactivation conventionally used, such as Tween-TNBP solvent/detergent treatment, which have been proven to be effective in reducing the viral load of blood derivatives, such as cryoprecipitated plasma proteins (factor VIII, von Willebrand factor, etc.). Given that the obtaining or the treatment of biological products, such as plasma clotting proteins, should incorporate viral elimination/inactivation steps in view of a therapeutic use, the pharmaceutical industry for blood-derived medicaments today seeks to evaluate the theoretical risk of transmission of variant CJD by blood-derived products.

Currently, the method for the titration of NCTA-related infectiousness conventionally employed uses an in vivo titration method (for example, the golden hamster for titrating infectiousness related to the 263k strain), by intracerebral injection of various dilutions of an NCTA-loaded test product. Depending on the number of animals affected in the various groups corresponding to the dilutions carried out, it is possible to calculate an infectious titre and to establish the reduction factor of a given process on the basis of an untreated reference. However, this method has the drawback of being long (approximately one year), expensive and relatively incompatible with industrial-scale development, which requires a result regarding prion elimination effectiveness as rapidly as possible.

In addition, it is often necessary to introduce a step for concentrating the infectious agent so as to increase the sensitivity of the titration methods. All the procedures for concentrating infectious agents responsible for TSE nowadays involve purification of PrPsc.

Other methods have been proposed for the in vitro titration of TSE infectious agents.

Techniques for detecting PrPsc by Western blotting (MacGregor, Transfusion J. Medicine 2001; 11, 3-14) or by ELISA generally require prior digestion of the sample to be analysed, with proteinase K, or denaturation with chaotropic agents in order to distinguish the pathological protein (PrPsc) from the normal protein (PrP).

Another titration method has recently been developed, based on the use of PrPsc-specific antibodies which do not recognise PrP (Korth et al., Nature 1997 Nov. 6, 390 (6655); 74-7).

U.S. Pat. No. 6,150,583 describes, for its part, the production of transgenic animals expressing a PrP labelled with a heterologous epitope which is or is not exposed at the surface of the prion protein, depending on the conformation of the latter.

Document WO 2005/022148 describes a method for the in vitro titration, called “TCIA” (“tissue culture infectivity assay”), of an NCTA in a biological product, by means of bringing stable transgenic cells which tolerate replication of said NCTA into contact with the biological product, and then culturing these cells for one or more passages in order to amplify the amount of NCTA present in the biological product by replication of the NCTA. More specifically, the TCIA consists in bringing successive dilutions of an infectious homogenate (scrapie strain 127-S) into contact with cells in culture on a multiwell plate, in a proportion of 5 wells per dilution. The number of positive wells is then evaluated by detection of PrPsc (marker indissociable from infectiousness) after 8 to passages, and the titre is determined by the Spearman-Karber method.

The process called “PMCA” (Protein misfolding cyclic amplification), described in the document Saborio et al. (Nature; 2001, 411, 810-3), envisages, for its part, bringing pathological forms originating from a tissue or a fluid derived from a contaminated animal into contact with a nonpathological form of the NCTA protein, in order to convert the latter into a pathological form.

However, the TCIA and PMCA processes have disadvantages. Thus, the in vitro TCIA titration method still proves to be long (10 weeks) for routine use. In addition, it is, at the current time, slightly less sensitive than the bioassay method involving the infection of a laboratory animal.

The method of PMCA, although still under development, is found to be at least as sensitive as the bioassay. However, it does not provide any evidence as to the infectiousness associated with the PrPsc detected and proves to be difficult to implement with plasma matrices. In addition, uncertain reproducibility and also false-positive results have been reported (TSE meeting, Cambridge Healthtech Institute's12^{th} annual, 2008 Feb. 11-12). There is still, therefore, a great need to develop a method for determining infectiousness which is applicable on an industrial scale, i.e., in particular: reproducible, compatible with various matrices (for example, blood derivatives), sensitive (with a sensitivity equivalent to the bioassay), and relatively rapid.

### SUMMARY OF THE INVENTION

The invention now provides an improved in vitro method for the in vitro detection and/or titration of a non-conventional transmissible agent (NCTA) or of a protein of pathological conformation, which is a marker for infectiousness related to the NCTA, in a sample. The method of the invention comprises, in an in vitro cell culture system, replication or propagation, in cells in culture or at their surface, of the NCTA present in the sample, and then repeated incubation with a substrate which allows amplification of the NCTA or of the protein of pathological conformation, before determination of the presence and/or of the amount of the NCTA or of the protein of pathological conformation in the sample.

The inventors have in fact demonstrated that it is possible, by combining the product resulting from the culturing of the cells with a source of substrate for the protein of pathological conformation (it being possible for the substrate to contain, for example, PrP) and/or the NCTA, to convert this substrate into a protein of pathological conformation (PrPsc) and/or into NCTA that can be detected and quantified.

More specifically, the invention is directed towards an in vitro method for detection and/or titration of a non-conventional transmissible agent (NCTA) or of a protein of pathological conformation which is a marker for the infectiousness of the NCTA, in a sample, comprising the steps consisting in:
i) bringing said sample into contact with cells which tolerate the replication or propagation of the NCTA,
ii) culturing said cells in order to replicate or propagate the NCTA present in said sample,
iii) bringing into contact and incubating the NCTA with a source of substrate for the protein of pathological conformation and/or the NCTA, by means of which the amount of protein of pathological conformation and/or of NCTA is amplified,
iv) disaggregating the aggregates possibly formed,
v) determining the presence and/or the amount of protein of pathological conformation and/or of NCTA in the sample,
   steps (iii) and (iv) constituting a cycle of operations which is repeated at least twice before step (v).

Preferably, the sample is chosen from the group constituted of blood products and derivatives thereof, food products and cosmetic products.

The invention is also directed towards an in vitro method for the evaluation and/or monitoring of a process for obtaining or treating a biological product or a material which may be contaminated with an NCTA, in which method a titration method as defined above is applied to said biological product or material, (A) upstream and (B) downstream of said process, and the two titre values (A) and (B) obtained are compared.

Another subject of the invention concerns an in vitro method for the evaluation and/or monitoring of a procedure for decontaminating a biological product or a material, in which method a titration method as defined above is applied to said biological product or material, (A) upstream and (B) downstream of said procedure, and the two titre values (A) and (B) obtained are compared.

Yet another subject of the invention is an in vitro method for the diagnosis of a transmissible spongiform encephalopathy in a human or nonhuman animal individual, which comprises the detection of the presence, in a biological sample from said individual, of a non-conventional transmissible agent (NCTA) or of a protein of pathological conformation, which is a marker for infectiousness of the NCTA, by means of the method as defined above.

Finally, another subject of the invention is an in vitro method for the evaluation of the ability of a compound to modulate (i.e. inhibit or increase) the infectiousness or the replication or the propagation of an NCTA.

The detection method of the invention makes it possible to detect amounts of NCTA which are at least equivalent to those detected by the known processes of the prior art, including the bioassay method, considered to be the reference method. Moreover, it makes it possible to obtain results more rapidly than the known methods of the prior art.

### FIGURE LEGEND

The FIGURE is an autoradiograph of a gel showing the detection of PrP^{sc} by means of the method of the invention. The lanes are the following:

1: molecular weight marker
2 to 12: lysates of cells inoculated with LN-3326 and harvested, respectively from passage 1 to 10
13: lysate of cells inoculated with LN-3777 (healthy brain homogenate)
14: homogenate of infected brain (LN-3326) diluted 10⁶ times.

### DETAILED DESCRIPTION OF THE INVENTION

### Definitions

In the context of the invention, the term “NCTA” represents any non-conventional transmissible agent, such as those responsible, in humans, for familial or sporadic CJD, for Kuru disease or for variant CJD, or alternatively those responsible, in animals, for natural TSEs, such as ovine scrapie, bovine or feline spongiform encephalopathy, chronic wasting disease in cervids or spongiform encephalopathy in mink, or, finally, TSE strains experimentally adapted to laboratory animals. The term “PrPsc” herein denotes the prion protein of pathological conformation (or pathological conformer). The “pathological” form of PrP is the form of the protein of which the conformation is correlated with the appearance of a TSE in infected human or nonhuman animals.

In the context of the invention, the NCTA is also denoted by the term “infectious agent”. The NCTA titrated by means of the method according to the invention is preferably of ovine, bovine, murine, cricetid, cervid, primate or human origin. Preferably, the NCTA may be the protein of pathological conformation itself.

The term “sample” denotes any source of material which may be contaminated with an NCTA. Such a source of material may, for example, be a liquid, a food product, a drink, a cosmetic product or a product derived from genetic engineering, a molecule capable of inhibiting the infectiousness of an NCTA, this list not being limiting. Preferably, it is a biological sample, for example a biological fluid or a tissue or tissue extract. In the case of a tissue, the method of the invention can be carried out on homogenates or directly on samples ex vivo. Such a tissue may be a brain tissue, a vertebral column tissue or a tonsil tissue, this list not being limiting. The sample may also be a composition derived from a human or animal source, such as growth hormones or cell extracts, for instance pituitary extracts. Such a composition can in fact be contaminated with an NCTA. In the case of a biological fluid, the latter may be blood, lymph, urine or milk, this list not being limiting. Preferably, the sample is a blood product or a derivative, for example a plasma derivative or a plasma protein concentrate.

The expression “cells which tolerate the replication or propagation of the NCTA”, as used in step (i), denotes any cell capable of being infected with an NCTA, of replicating or propagating this NCTA and of producing PrPsc and/or infectiousness. These cells are preferably cells in which the gene encoding the nonpathological conformer of the PrP prion protein has been integrated and/or overexpressed. More preferably, these cells are stable transgenic cells which are capable of expressing PrP.

A “passage” is generally the subculturing of a part of the cells of a culture dish in another dish containing new culture medium. Each passage therefore makes it possible to dilute the cells which no longer have space to divide, in another dish, and the period of culture in a dish enables the NCTA to replicate.

The expression “source of substrate for the NCTA” denotes any noninfectious matrix capable of supporting the replication of the NCTAs via in vitro amplification cycles.

The expression “source of substrate for the protein of pathological conformation” denotes any source of protein of nonpathological conformation, which cannot modify the conformation of another protein so as to make it insoluble. Preferably, the source of substrate for the NCTA and/or for the nonpathological normal form of the protein can belong to the same species, or to a species different from the biological product tested. This source may, for example, be derived from a nonpathological normal form of the protein of the same product as that contained in the test sample. Alternatively, the source of nonpathological normal form may be produced recombinantly or by synthesis, using means known to those skilled in the art.

### Cells of Step (i):

The cells used in step (i) of the method of the invention advantageously exhibit the following criteria:
compatibility between the speed of replication or multiplication of the cells and the incubation time necessary to demonstrate replication of the NCTA in the infected cells;
stability of the cells after they have been brought into contact with the infectious agent, which can be demonstrated by the absence of cytotoxicity related to the accumulation of the NCTA in the cells;
good sensitivity of the cells to infection, evaluated, for example, through the low multiplicity of infection for obtaining accumulation of the NCTA in the cells exposed to the infectious agent.

Given that nerve cells (neurons, glial cells) are not necessarily the best substrates for the purposes of in vitro titration, owing to their poor adaptation in culture, it is preferred to establish transgenic cell lines by combination of a specific cell type and a particular transgene, by means of known techniques, necessary for providing an ideal environment for replication of the NCTA.

In one preferred embodiment, the cells of step (i) are rabbit epithelial cells, in particular cells of the Rov9 line (Vilette et al. PNAS, March 2001; 98; 4055-9).

Vilette et al. have shown that stable, transgenic epithelial cells from rabbit, which express ovine PrP (transgene), are capable of replicating a scrapie strain and of producing ovine PrPsc after infection with an infectious brain homogenate containing ovine PrPsc. The cell model constructed, the Rov9 line, inducibly expresses the exogenous PrP, which guarantees the maintaining thereof during the incubation period necessary for the accumulation of PrPsc in these cells brought into contact with an infectious material.

In another embodiment, the cells of step (i) are murine glial cells, in particular cells of the MovS2 or MovS6 line (Archer et al. Journal of Virology, 2004; 78 p. 482-90). These lines consist of murine glial cells which express ovine PrP and tolerate the replication of NCTA of ovine origin.

Bringing into Contact with the Test Sample:

The cells which tolerate the replication or propagation of the NCTA are brought into contact with the test sample that may be infected with this NCTA, or with an infectious material containing the NCTA as reference material, for example extracts of brains from animals infected with an NCTA, such as an ovine prion. The bringing into contact is carried out according to methods known to those skilled in the art (see, for example, Archer et al. Journal of Virology, January 2004, p. 482-490).

These cells are then cultured for one or more passages in order to allow the NCTA to replicate or to propagate.

Advantageously, the transgenic cells can be brought into contact with at least one dilution of the sample that may be infected with the NCTA, in a biologically acceptable aqueous solution, in particular with several dilutions, most particularly with serial or successive dilutions. These dilutions make it possible to refine the quantification of the infectiousness in the sample tested.

Several cell replicates can be brought into contact with the same dilution of the biological product, in order to give the results a finer statistical resolution.

### Cell Culture (Step ii):

The step of culturing the cells potentially infected with the biological product, according to any technique known to those skilled in the art, is required for the replication or propagation of the NCTA, and therefore for amplifying an amount of NCTA which is initially insufficient to be detected.

In one exemplary embodiment, step ii) of culturing stable transgenic cells, performed so as to amplify the amount of NCTA present in said biological sample by replication of the NCTA, is carried out in DMEM+Ham-F12 (4:1) medium supplemented with glutamine and foetal calf serum (5% final concentration). The cells are incubated at 37° C. under 5% CO₂. One passage of the cells (split ratio of: 1 to 10) is carried out each week.

The product resulting from culturing the cells contains the marker protein (in particular PrP) in its pathological form, the amount of which is greater than the amount initially present in the biological sample, if said sample contains same. The marker protein (in particular PrP) in its pathological form and the NCTA accumulate in the cell culture (at the level of the infected cells and in the culture medium).

Bringing into Contact with a Source of Substrate for the Protein of Pathological Conformation and/or the NCTA (Step iii):

Step ii) of the method of the invention is followed by bringing into contact and incubating the product resulting from the culturing of the cells, with a source of substrate for the pathological conformer and/or for the NCTA.

This source of substrate can be provided, for example, in the form of a product of animal origin, for example a healthy brain homogenate, or else of material derived from in vitro culture. This material can, for example, be derived from cells, such as MovS, or Rov N2A (Weissmann et al. (2003), PNAS Vol. 100 No. 20 p. 1666-11671), or else from yeast, from mycetes or from bacteria, this list not being limiting. This material derived from in vitro culture can be expressed in various cell compartments, such as the extracellular compartment (for example the supernatant, exosomes, this list not being limiting) and/or membrane and/or cytosolic compartments (cell lysate, for example). The function of this step is to allow the in vitro amplification of the protein of pathological conformation (in particular PrPsc) and/or of the NCTA harvested during step ii) by conversion of the non-pathological form of the marker protein (in particular PrP) (contained in the substrate) into pathological form, autoconversion of the nonpathological form being theoretically impossible. The protein in its pathological form would initiate the transformation of the nonpathological form into the pathological form.

At the end of the conversion reaction, the unconverted nonpathological form is not detected by the detection system, as will be explained below.

The incubation of the product of the cell culture of step (ii) with a source of substrate for the protein of pathological conformation and/or the NCTA is carried out for a period of time sufficient to allow at least a part of the proteins which have a nonpathological form to be transformed into a pathological form of the protein, or to allow the NCTA to amplify. Preferably, each incubation step is carried out for a period of time of between 10 seconds and 4 hours, preferably between 20 minutes and 1 hour, and particularly preferably for 30 minutes.

The amplification medium can advantageously be constituted of a buffer typically composed of: (1×PBS), 150 mM NaCl and 1% Triton, and at least of a substrate containing the marker protein (in particular PrP) in a nonpathological conformation (for example, of a volume 10 times greater than the volume of the sample to be amplified).

### Aggregate Separation:

It is found that the proteins converted into pathological forms (of PrP^{sC} type) can aggregate with one another and with other particles of pathological form (PrP^{sC}), preventing the conversion of other proteins of nonpathological form into a pathological form. This is a drawback since the method could thus be slowed down by the low number of “conversion foci” present in the reaction medium.

Thus, step iv) of the method of the invention consists of the disaggregation of the aggregates possibly formed, so as to release the particles of marker proteins of pathological conformation and/or of NCTA so that they can convert other nonpathological proteins.

Many methods can be used to disaggregate the aggregates during step iv) of the method of the invention. By way of example, mention may be made of treatment with a solvent (such as sodium dodecyl sulphate, dimethyl sulphoxide, acetonitrile, guanidine, urea, trifluoro-ethanol, dilute trifluoroacetic acid, dilute formic acid, this list not being limiting), modification of the physicochemical characteristics of the solution, such as the pH, the temperature, the ionic strength or the dielectric constant, and also physical methods, such as sonication, laser irradiation, freezing/-thawing, autoclave incubation, high pressure, mild homogenisation, or alternatively other sources of irradiation, this list not being limiting. Sonication is preferably used. Sonication is a method known to those skilled in the art and often used in methods for purifying PrP^{sc}, by making it possible to increase aggregate solubility.

It is possible that not all the aggregates will be disaggregated during the implementation of a single disaggregation step. In this case, the concentration of pathological proteins increases over the course of the disaggregation steps.

The duration of the disaggregation step can be readily determined by those skilled in the art, and it can depend on the disaggregation method selected. Preferably, the duration of the disaggregation step is between 1 second and 60 minutes, more preferably between 5 seconds and 30 minutes, and more particularly between 5 seconds and 30 seconds.

Advantageously, the succession of steps iii) and iv), called a cycle, is repeated at least twice, preferentially between 5 and 100 times, preferably between 20 and 60 times.

This cycle repeated between 2 and 100 times constitutes a series of amplification cycles. A series of cycles can also be repeated several times. In this case, the new series will be initiated starting from a volume of the preceding series in place of the initial NCTA sample.

### Protein Detection:

Step (v) of detecting the pathological proteins can be carried out by any method known to those skilled in the art.

The specific detection of PrPsc can be carried out by means of a first step of separating the two isoforms PrPc and PrPsc. This separation is carried out on the basis of biochemical properties of PrPsc which make it possible to distinguish it from the nonpathological proteins, in particular the fact that PrPsc is reportedly more resistant to protease-based treatments and less soluble even in the presence of detergents. Thus, the first step after the amplification is preferably the separation of PrPc (nonpathological soluble normal form of PrP) from the sample, which can be carried out for example by means of protease treatment, for example proteinase K treatment, or by centrifugation in order to separate the soluble forms (PrPc) from the insoluble forms (PrPsc).

In one particular embodiment, the digestion with proteinase K is a step prior to Western blotting, it digests mainly PrPc and not PrPsc. It is in fact a property of PrPsc that it is relatively resistant to PK compared with PrPc. The subsequent detection step therefore no longer detects the nonpathological form of the protein since said form has been digested by the protease.

The detection step can be carried out using the following methods: immunocytochemistry (for example by cell labelling and Facscan analysis) or immunochemistry (such as Western blotting or an ELISA assay), or a radioactivity assay, a fluorescence assay, an electron-microscopy assay, a turbidimetry test for detecting the aggregates, and also structural tests including NMR (nuclear magnetic resonance), circular dichroism, Raman spectroscopy, UV absorption, a monoclonal antibody which recognises the pathological form of the protein, this list not being limiting.

It is also possible to detect the pathological form of the proteins by means of an antibody which specifically recognises the pathological form and not the nonpathological form. This antibody can itself be labelled in order to make it easier to detect. Such an antibody may, for example, be the 15B3 antibody (Korth et al., 1997, Nature 1997 Nov. 6, 390 (6655): 74-7).

### NCTA Titration:

The detection of the pathological forms of the marker proteins or of the NCTA can be combined with a determination of the amount of these proteins or of the NCTA present in the sample.

The titration can be carried out by means of any titration method known to those skilled in the art. In particular, it can be carried out according to the model of the methods described in documents WO2005022148 and WO2006117483 (in particular, the reference examples A and B).

All the results of Western blotting for the various dilutions and the various replicates can be analysed by means of a statistical method known to those skilled in the art which makes it possible to establish an infectious titre, for instance the Spearman-Karber method (Schmidt N. J., Emmous R. W., Diagnostic Procedures for viral, ricketsial and chlaveydial Infection, 1989, 6^{th} edition).

In one embodiment of the invention, the method for calculating the titre is the Spearman-Karber method. This method assumes dilution of the test sample according to a geometric progression, i.e. in a constant proportion between the successive dilutions, and inoculation of a constant volume (in general 0.150 ml) of each dilution in at least five wells. The dilution factor most commonly used is the decimal factor.

In order for the Spearman-Karber formula to be applicable, it is necessary to use a constant number of wells inoculated with each dilution, a constant dilution factor and a range of dilutions which is sufficiently broad to encompass both the dilutions on either side of which one hundred percent of the wells will give a positive reaction, and the dilutions on either side of which one hundred percent of the wells will give a negative reaction.

If one or more of these conditions are not met, it is sometimes assumed that, for a constant dilution factor, the higher or lower dilution coming after the final dilution carried out would have given the desired result. Such a “fabrication” of data is not based on any theoretical foundation, but if it is applied with sufficient care, it is not dangerous. However, it is preferable to repeat the titration with a more suitable range of dilutions, which is essential if there are serious gaps in the data.

According to the Spearman-Karber formula:

Log₁₀ median dose=(X₀)−(d/2)+dS(rᵢ/nᵢ)

where:
X₀=log₁₀ of the reciprocal value of the lowest dilution at which all the test inocula are positive.
d=log 10 of the dilution factor, also referred to as “dilution step” (i.e. the difference between the dilution logarithm intervals).
nᵢ=number of test inocula used at each dilution.
Ri=number of positive test inocula (out of ni).
S(rᵢ/nᵢ)=S(P)=sum of the proportion of positive tests beginning at the lowest dilution giving one hundred percent of positive results.

The summation begins at the X₀ dilution.

The estimated standard deviation is calculated using the following formula:

Log standard deviation=d*√(E(p*(1−p)/(ni−1)))

With p=rᵢ/nᵢ=proportion of positive tests (i.e. of wells of inoculum exhibiting a reaction) at each dilution.

The method of the invention makes it possible to quantify the NCTA-related infectiousness over a range of approximately 4 log, i.e. of 10 000 in vitro infectious units. This can, for example, make it possible to meet the criteria for validation of the effectiveness of processes for obtaining biological products with respect to the elimination of NCTAs.

### Applications of the Method for Determining NCTA-Related Infectiousness:

The invention also relates to the application of the titration method according to the invention in a method for the in vitro evaluation and/or monitoring of a method for obtaining or treating a biological product which may be contaminated with an NCTA. This evaluation and/or monitoring method is characterized in that a titration method according to the invention, as described above, is applied to the biological product, upstream and downstream of said process, and the two titre values obtained are compared. By comparison between the two measurements, the degree of elimination of the NCTA or the NCTA reduction factor is determined.

In particular, the titration method according to the invention has the capacity to be readily applicable in any type of process for obtaining or purifying biological products, in particular blood products, such as blood plasma derivatives, using, for example, chromatographies or nanofiltration, in particular the chromatographies described in documents EP 0 359 593 and WO 02/092632 or the nanofiltration described in document WO 2005/022148.

Thus, the implementation of the method of the invention makes it possible to evaluate and/or monitor the effectiveness of a process (or of a part of a process) for obtaining or treating, or even purifying, any biological product which may be contaminated with an NCTA, in eliminating this NCTA, by means of a titration using specific transgenic cell lines which promote the replication of the NCTA, brought into contact with an infectious or potentially infectious material containing the NCTA to be tested. The amounts of NCTA are measured upstream and downstream of the process (or of the part of the process) of which it is desired to assess the effectiveness with regard to the NCTA. By comparing the two measurements, the degree of elimination of the pathogenic agent is determined. Thus, the implementation of the present method can be carried out during a process for obtaining a biological product or in the context of a treatment for eliminating the NCTA following the obtaining of the biological product.

The invention also concerns the application of the titration method according to the invention in a method for the in vitro evaluation and/or monitoring of a procedure for decontaminating a material. In this case, the NCTA titre of a biological product containing an NCTA is determined by means of the titration method according to the invention. This infected biological product is then brought into contact with the decontamination material and then the decontamination procedure is applied to this material. Finally, the titre of the biological product having undergone the decontamination procedure is again determined. The two titre measurements carried out upstream and downstream of the decontamination procedure are compared so as to evaluate the effectiveness of the decontamination procedure. The material may, for example, be a purification material, in particular a chromatography column, or else be the sanitising of a chromatography column using sodium hydroxide.

The invention also relates to the application of the titration method according to the invention in a procedure for selecting and/or method for evaluating the capacity of a candidate compound for modulating (reducing or increasing) the titre of the infectious material. In this case, the NCTA titre of a biological product containing an NCTA is determined by means of the titration method according to the invention. This infected biological product is then brought into contact with the test compound and then the titre of the biological product having undergone the selection procedure and/or evaluation method is again determined. The two titre measurements carried out upstream and downstream of bringing the sample into contact with the test compound are compared.

The invention also relates to the application of the titration method according to the invention in a procedure for selecting and/or method for evaluating a compound capable of inhibiting the infectiousness of an NCTA. In this case, the NCTA titre of a biological product containing an NCTA is determined in the presence and then in the absence of the compound to be evaluated, by means of the titration method according to the invention. The methods of bringing into contact the compound are determined according to whether the action prevents the initiation of an infectious cycle or blocks an already initiated infectious cycle. In any event, the titre of the biological product is determined with and without treatment with the test product. The two titre measurements carried out are compared in order to evaluate the inhibitory activity of the compound on the infectiousness of an NCTA.

The invention also relates to the application of the titration method according to the invention in a method for identifying a compound which makes it possible to modulate the transformation of the nonpathological form into the pathological form of a marker protein or of the NCTA, for example the transformation of PrP to PrP'. In this case, the NCTA titre of a biological product containing an NCTA is determined by means of the titration method according to the invention. This infected biological product is then brought into contact with the test compound and then the titration method of the invention is applied in order to again determine the titre of the biological product. The two titre measurements carried out upstream and downstream of the bringing into contact with the compound are compared in order to evaluate the effectiveness of the test compound.

The method of the invention will be understood more clearly with the aid of the additional description which follows, which does not limit the scope of the invention.

### EXAMPLES

### Materials and Methods

MovS6 cells (Archer F. et al., 2004, above) were selected as cells which tolerate the replication of NCTAs, and the 127-S scrapie strain adapted to Tg301 transgenic mice was used as natural infectiousness source (Vilette J L et al., 2001, above). The Tg301 transgenic mice carry a large DNA fragment isolated from an ovine artificial chromosome library, and the levels of expression of ovine PrP are approximately 8 times higher than the level observed in sheep brain. The initial infectiousness source consisted of a homogenate of brains (batch LN-3326) from infected mice at 200 mg/ml. The titre of this sample is 5.7 log₁₀ uWB/ml and 6.35 TCID₅₀/ml.

### Cell Culture and Inoculation:

The MovS6 cells were cultured in DMEM+Ham-F12 (4:1) medium supplemented with glutamine and foetal calf serum (5% final concentration), and incubated at 37° C. under 5% CO₂. Each week, 10% of the cells were re-seeded in each well. The remaining 90% of cells were either stored in the form of a dry cell pellet washed beforehand with PBS, or eliminated.

Titration plates were prepared 24 hours before the inoculation. The cells were seeded in wells of approximately 2 cm² (24-well plate format), in a proportion of 100 000 cells per well, i.e. approximately 50 000 cells/cm².

The inoculum consisted either of the LN-3326 sample diluted 10⁴-fold, or of a sample of healthy brain homogenate which served as a “negative control” (batch: LN-3777). The inoculations were carried out in 5 replicates.

The cells were brought into contact with 150 μl of diluted inoculum for 24 hours, and then 1 ml of new culture medium was added. The cells were maintained in culture for a further 72 hours, i.e. until the first passage, during which, for each well, the entire cell layer was transferred into a well of approximately 9.6 cm² (6-well plate format). The cell culture was then maintained for 10 weeks. Each week, for each well, the culture medium was changed and 10% of the cells were re-seeded. Of the cells inoculated with the infected inoculum (LN-3326) which were non-reseeded, 2 replicates out of the 5 were washed once in PBS and then stored at −80° C. in the form of a dry pellet (samples intended for amplification according to the method of the invention) and the other 3 replicates were conserved, without prior washing, as a dry pellet (samples intended for detection by WB). Of the cells inoculated with the healthy sample and not re-seeded, all the replicates were stored, without prior washing, in the form of a dry pellet. The cell pellets obtained made it possible to carry out the analyses described hereinafter.

### Detection of PrPsc in the Cells:

The cell pellets, stored at each passage, made it possible to investigate the PrPsc produced by the cells. The detection of the PrPsc produced was carried out either directly by Western blotting for the non-washed cell pellet samples, or by Western blotting after amplification by means of the method of the invention for the washed cell pellet samples.
Detection directly by Western blotting: The non-washed cell pellet samples (3 replicates out of the 5) were thawed and taken up in 60 μl of PBS. The cells were lysed by sonication for 15 seconds using a sonication bath (power: 15W). 20 μl of sonicated cell lysate were removed in order to be treated with proteinase K. The digestion product was denatured and then analysed by polyacrylamide gel electrophoresis under denaturing conditions (SDS-PAGE). The proteins having migrated in the gel were transferred onto a PVDF membrane by electroblotting. The PrPsc present on the membranes was detected by incubation with the 6H4 antibody (Prionics) and then a secondary antibody labelled with alkaline phosphatase (“anti-mouse antibody” goat antibody). The labelled membranes were revealed by chemiluminescence. A sample was considered to be positive if the electrophoretic profile of the three forms of glycosylated PrPsc was visible on the autoradiograms.
Detection by means of the method of the invention: Each of the washed cell pellet samples was thawed and then taken up in 90 μl of 5% healthy brain homogenate solution and placed in a 200 μl microtube. A cycle of steps (iii) to (v) was composed of an incubation phase for 30 minutes at a temperature of 37° C. followed by a sonication phase for 20 seconds. A series of 50 cycles was carried out on the samples using the automated Misonix sonicator 3000 (power level set at 7). Using 18 μl of amplification product, digestion with proteinase K followed by analysis by Western blotting was carried out in order to detect the possible presence of PrPsc.

### Results

The detection of PrPsc in the various samples is represented in the FIGURE and summarised in Table 1.

TABLE 1
Number of positive wells after detection of
PrPsc in the MovS6 cell lysates

| Cells | PrPsc | | | | | | | | | | |
|---|---|---|---|---|---|---|---|---|---|---|---|
| inoculated | detection | Passage | | | | | | | | | |
| with | method | 1 | 2 | 3 | 4 | 5 | 6 | 7 | 8 | 9 | 10 |
| LN-3326* | Western | 0/3 | 0/3 | 0/3 | 3/3 | 3/3 | 3/3 | 3/3 | 3/3 | 3/3 | 3/3 |
| | blotting | | | | | | | | | | |
| LN-3326 | PMCA + | 1/1 | 1/1 | 1/1 | 1/1 | 1/1 | 1/1 | 1/1 | 1/1 | 1/1 | 1/1 |
| | Western | | | | | | | | | | |
| | blotting | | | | | | | | | | |
| LN-3777** | Western | 0/3 | 0/3 | 0/3 | 0/3 | 0/3 | 0/3 | 0/3 | 0/3 | 0/3 | 0/3 |
| | blotting | | | | | | | | | | |
| LN-3777 | PMCA + | NT | NT | 0/1 | NT | NT | NT | NT | NT | NT | NT |
| | Western | | | | | | | | | | |
| | blotting | | | | | | | | | | |

*LN-3326: inoculum infected with the 127S scrapie strain
**LN-3777: noninfected inoculum
NT: not tested

No PrPsc signal was detected in the sample consisting of healthy brain homogenate. Likewise, no signal was observed in the lysate of MovS6 cells inoculated with this healthy brain homogenate (FIGURE, wells 2 and 13).

A signal specific for PrPsc was observed in the sample consisting of homogenate of brain infected with the 127-S scrapie strain, diluted 10⁶-fold (FIGURE, well 14). Given the titre of 5.7 log¹⁰ uWB/ml of this homogenate, and the detection limit of the Western blotting method of 2.36 log¹⁰ uWB/ml, the PrPsc in this diluted sample was not detected solely by the Western blotting method.

A signal specific for PrPsc was observed in the lysates of MovS6 cells inoculated with the homogenate of brain infected with the 127-S strain. This signal was observed as early as the first passage (FIGURE, wells 3 to 12).

### CONCLUSIONS

Amplification of the PrPsc Related to the 127-S Strain in the LN-3326 Brain Homogenate:

These data show that the PrPsc associated with the 127-S strain in the sample of LN-3326 brain homogenate prediluted 10⁶-fold can be amplified by means of the method of the invention. The detection of PrPsc in this diluted sample indicates that the degree of amplification is at least 2.5 to 3 log₁₀.

Moreover, this amplification is PrPsc-specific since no signal is observed with the nondiluted sample of healthy homogenate.

Amplification of the PrPsc Associated with the 127-S Strain in the Cell Lysates:

At the dilution studied (10⁻⁴), the LN-3326 homogenate induced the production of PrPsc detectable by Western blotting starting from the 4^{th} passage. This delay corresponds to the time necessary for the propagation of the PrPsc in the cell culture to reach the minimum concentration level detectable by Western blotting. This delay also proves that there is de novo PrPsc production by the cells.

With the method of the invention, the PrPsc present in the infected cells is detected as early as the first passage and for all the subsequent passages. This amplification appears to be specific since PrPsc was not detected in the noninfected cells.

Thus, these data appear to indicate that the method of the invention, compared with the Western blotting method, allows earlier detection of PrPsc produced by the infected cells.

1. In vitro method for the detection and/or titration of a non-conventional transmissible agent (NCTA) or of a protein of pathological conformation which is a marker for infectiousness of the NCTA, in a sample, comprising the steps consisting in:
i) bringing said sample into contact with cells which tolerate the replication or propagation of the NCTA,
ii) culturing said cells in order to replicate or propagate the NCTA present in said sample,
iii) bringing into contact and incubating the NCTA with a source of substrate for the protein of pathological conformation and/or the NCTA, by means of which the amount of protein of pathological conformation and/or of NCTA is amplified,
iv) disaggregating the aggregates possibly formed,
v) determining the presence and/or the amount of protein of pathological conformation and/or of NCTA in the sample,
steps (iii) and (iv) constituting a cycle of operations which is repeated at least twice before step (v).

2. The method according to claim 1, in which the protein of pathological conformation, which is a marker for the NCTA, is the PrP^{sc} prion protein.

3. The method according to claim 2, in which the cells of step (i) are cells in which the gene encoding the nonpathological conformer of the PrP prion protein has been integrated.

4. The method according to claim 1, in which the determination of the presence and/or of the amount of the protein of pathological conformation in step (v) is carried out by immunochemistry or immunocytochemistry.

5. The method according to claim 1, in which the cells are rabbit epithelial cells, in particular cells of the Rov9 line.

6. The method according to claim 1, in which the cells are murine glial cells, in particular cells of the MovS2 or MovS6 line.

7. The method according to claim 1, in which said cycle of steps (iii) and (iv) is repeated from 5 to 60 times before step (v).

8. The method according to claim 1, in which the sample is selected from the group consisting of blood products and derivatives thereof, food products and cosmetic products.

9. The method according to claim 1, in which the substrate for the pathological form of the protein and/or for the NCTA is provided in the form of a healthy brain homogenate.

10. An In vitro method for the evaluation and/or monitoring of a process for obtaining or treating a biological product or a material which may be contaminated with an NCTA, in which method a titration method according to claim 1 is applied to said biological product or material, (A) upstream and (B) downstream of said process, and the two titre values (A) and (B) obtained are compared.

11. An In vitro method for the evaluation and/or monitoring of a procedure for decontaminating a biological product or a material, in which method a titration method according to claim 1 is applied to said biological product or material, (A) upstream and (B) downstream of said procedure, and the two titre values (A) and (B) obtained are compared.

12. An In vitro method for evaluating the ability of a compound to modulate the infectiousness of an infectious biological product, in which method a titration method according to claim 1 is applied to said infectious biological product, (A) in the presence and (B) in the absence of said compound to be evaluated, and the two titre values (A) and (B) obtained are compared.

13. An In vitro method for the diagnosis of a transmissible spongiform encephalopathy in a human or nonhuman animal individual, which comprises the detection of the presence, in a biological sample from said individual, of a non-conventional transmissible agent (NCTA) or of a protein of pathological conformation which is a marker for infectiousness of the NCTA, by means of the method as defined in claim 1.

14. The method according to claim 2, in which the determination of the presence and/or of the amount of the protein of pathological conformation in step (v) is carried out by immunochemistry or immunocytochemistry.

15. The method according to claim 3, in which the determination of the presence and/or of the amount of the protein of pathological conformation in step (v) is carried out by immunochemistry or immunocytochemistry.

16. The method according to any one of claim 2, in which the cells are rabbit epithelial cells, in particular cells of the Rov9 line.

17. The method according to any one of claim 3, in which the cells are rabbit epithelial cells, in particular cells of the Rov9 line.

18. The method according to any one of claim 4, in which the cells are rabbit epithelial cells, in particular cells of the Rov9 line.

19. The method according to any one of claim 14, in which the cells are rabbit epithelial cells, in particular cells of the Rov9 line.

20. The method according to any one of claim 15, in which the cells are rabbit epithelial cells, in particular cells of the Rov9 line.
